(19) United States
(12) Patent Application Publication
(10) Pub. No.: US 2024/0374482 A1
(43) Pub. Date: Nov. 14, 2024

(54) TOOTHPASTE DELIVERY SYSTEM

(71) Applicant: Fernando Ferreira, Cranston, RI (US)

(72) Inventor: Fernando Ferreira, Cranston, RI (US)

(21) Appl. No.: 18/662,233

(22) Filed: May 13, 2024

Related U.S. Application Data
(60) Provisional application No. 63/465,684, filed on May 11, 2023.

(51) Int. Cl. A61K 8/02; A61K 8/73; A61Q 11/00

(52) U.S. Cl. CPC A61K 8/0204; A61K 8/73; A61Q 11/00

(57) ABSTRACT

A toothpaste delivery system featuring a dissolvable packet filled with powdered toothpaste placed on a brush that releases the toothpaste into the user's mouth while brushing. The toothpaste delivery system having a front layer joined at its seams with a rear layer that defines a thin rectangular structure. The toothpaste delivery system is composed of consumable and dissolvable materials. The interior volume of the toothpaste delivery system includes a powdered dentifrice, which includes carbon activated particles or other substances that enhance the properties of the powdered dentifrice. The method for using the toothpaste delivery system includes the steps of placing the toothpaste delivery system in between the bristles of a toothbrush. When the toothbrush is then wet by the user, the toothpaste delivery system begins to dissolve and continues to do so as the user brushes to release the powdered dentifrice.

### CROSS-REFERENCE TO RELATED APPLICATIONS

This is a U.S. Non-Provisional Patent Application that claims the benefit of co-pending U.S. Provisional Patent Application No. 63/465,684 filed May 11, 2023, which is incorporated herein by reference in its entirety.

### FIELD OF THE INVENTION

The present disclosure relates to a toothpaste system and, more particularly, to a toothpaste delivery system.

### BACKGROUND OF THE INVENTION

Toothpaste may come as a powder or in a standard tube, as a paste or gel. When applying the standard forms of toothpaste upon a toothbrush, the toothpaste may fall on a countertop or on the floor. Moreover, it may be difficult to clean-up the standard forms of toothpaste when they fall onto undesirable surfaces. Thus, the standard forms of toothpaste may be messy. Additionally, when individuals apply the standard forms of toothpaste, they often apply too much upon the toothbrush. Using an excessive amount of toothpaste may be wasteful because only a small amount of toothpaste is required to clean a user's mouth effectively and efficiently. Also, when the user gets to the end of the standard tube, it may be difficult to get all the toothpaste out of the tube. This may leave users to discard the remaining toothpaste they may not extract from the tube, creating additional waste of the toothpaste.

Another form of toothpaste that is used are chewable toothpaste tablets. The chewable toothpaste tablets may not evenly distribute toothpaste within the user's mouth and along their teeth. This will leave areas of the user's mouth without the proper care. Moreover, the chewable toothpaste tablets may cause discomfort to the user. Chewing on the toothpaste tablet may cause the user's teeth to become hypersensitive and may cause subsequent damage to the user's teeth.

In order to address these concerns, the present invention provides users with a toothpaste delivery system featuring a front layer and a rear layer filled with powdered denitrifies placed on a brush that releases the toothpaste into the user's mouth while brushing.

### BRIEF SUMMARY OF THE INVENTION

The invention itself and manner in which it may be made and used may be better understood after a review of the following description, taken in connection with the accompanying figures.

The present invention is toothpaste delivery system, comprising a front layer, a rear layer, a plurality of seams joining the front layer and the rear layer, an interior volume formed between the front layer and the rear layer, and wherein the toothpaste delivery system is adapted to be sandwiched in between a plurality of bristles of a toothbrush.

It is an object of the present invention to provide a toothpaste delivery system that the bristles of the toothbrush are placed under water to dissolve the toothpaste delivery system onto the bristles of the toothbrush.

It is an object of the present invention to provide a toothpaste delivery system that the bristles of the toothbrush with the toothpaste delivery system are adapted to be placed into a user's mouth, so the toothpaste delivery system will continue to dissolve.

It is an object of the present invention to provide a toothpaste delivery system that the bristles of the toothbrush with the toothpaste delivery system are adapted to be placed into the user's mouth and the toothpaste delivery system will release the powdered dentifrice.

It is an object of the present invention to provide a toothpaste delivery system that when a user is adapted to brush their teeth with the toothbrush and the front layer and the rear layer of the toothpaste delivery system is dissolved, the powdered dentifrice spreads onto the bristles of the toothbrush and onto a user's teeth.

It is an object of the present invention to provide a toothpaste delivery system that the user completes brushing their teeth, they will rinse their mouth and the toothbrush to clean away remnants of the toothpaste delivery system.

### BRIEF DESCRIPTION OF DRAWINGS

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 shows a perspective view of an alternative embodiments of the toothpaste delivery system in use.

FIG. 2 shows a perspective view of alternative embodiments of the toothpaste delivery system.

FIG. 3 shows a perspective view of alternative embodiments of the toothpaste delivery system.

FIG. 4 shows a perspective view of alternative embodiments of the toothpaste delivery system in use.

FIG. 5 shows a perspective view of an embodiment of the toothpaste delivery system in use.

FIG. 6 shows an overhead view of a dental pouch of the toothpaste delivery system.

### DETAILED DESCRIPTION OF THE INVENTION

The non-limiting exemplary embodiment(s) will now be described more fully hereinafter with reference to the accompanying drawings, in which a preferred embodiment of the disclosure is shown. Such exemplary embodiment(s) may, however, be embodied in many different forms and should not be construed as limited to the embodiment set forth herein. Rather, these embodiment(s) are provided so that this application will be thorough and complete, and will fully convey the true scope of the disclosure to those skilled in the art.

The below disclosed subject matter is to be considered illustrative, and not restrictive, and any appended claim(s) are intended to cover all such modifications, enhancements, and other embodiment(s) which fall within the true scope of the non-limiting exemplary embodiment(s). Thus, to the maximum extent allowed by law, the scope of the non-limiting exemplary embodiment(s) is to be determined by the broadest permissible interpretation of the claim(s) and their equivalents and shall not be restricted or limited by the foregoing detailed description.

References in the specification to “an exemplary embodiment”, “an embodiment”, “a preferred embodiment”, “an alternative embodiment”, and similar phrases mean that a particular feature, structure, or characteristic described in connection with the embodiment(s) is included in at least an embodiment of the disclosure. The appearances of the phrase “a non-limiting exemplary embodiment” in various places in the specification are not necessarily all meant to refer to the same embodiment.

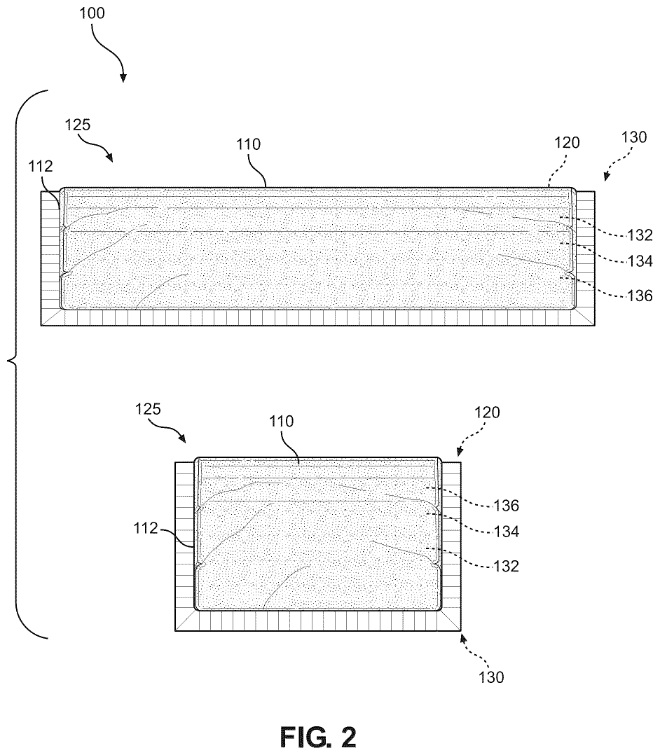

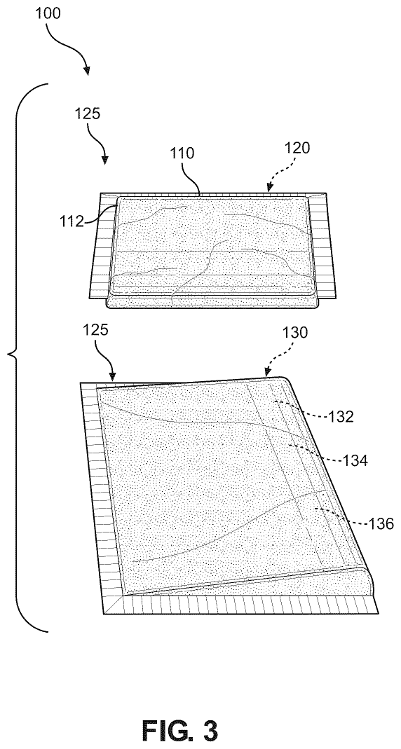

Referring now to FIGS. 2 and 3, there are shown perspective views of alternative embodiments of a toothpaste delivery system 100.

The toothpaste delivery system 100 may comprise a front layer 110 and a rear layer 120.

The front layer 110 may be joined to the rear layer 120 with a plurality of seams 112, defining a relatively thin rectangular structure, a relatively thin square structure, or the like. More specifically, the front layer 110 and the rear layer 120 may form a dental pouch 125. The dental pouch 125 may be composed of consumable and dissolvable materials or the like. In one embodiment of the toothpaste delivery system 100, the dental pouch 125 may be composed of Pullulan or the like. In one embodiment of the dental pouch 125 may be made of calcium-based or carbon-based material or the like.

The front layer 110 and the rear layer 120 may be made from a film used to create the dental pouch 125.

The toothpaste delivery system 100 initially included during its reduction to practice a plurality of dissolvable dental strips (not shown) to make the dental pouch 125.

The toothpaste delivery system 100 may further comprise an interior volume 130. The interior volume 130 of the toothpaste delivery system 100 may include a powdered dentifrice 132 or the like, which may include carbon activated particles 134 or other substances that enhance the properties of the powdered dentifrice 132. The toothpaste delivery system 100 contains a predetermined amount of the powdered dentifrice 132 which limits the use of excessive toothpaste products. The toothpaste delivery system 100 may further include a plurality of additives 136. The additives 136 that may be included are for freshening breath, teeth whitening, or any other additives associated with oral care.

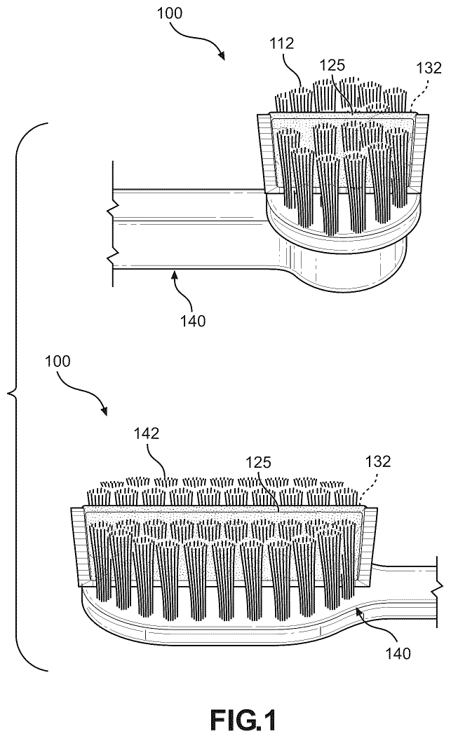

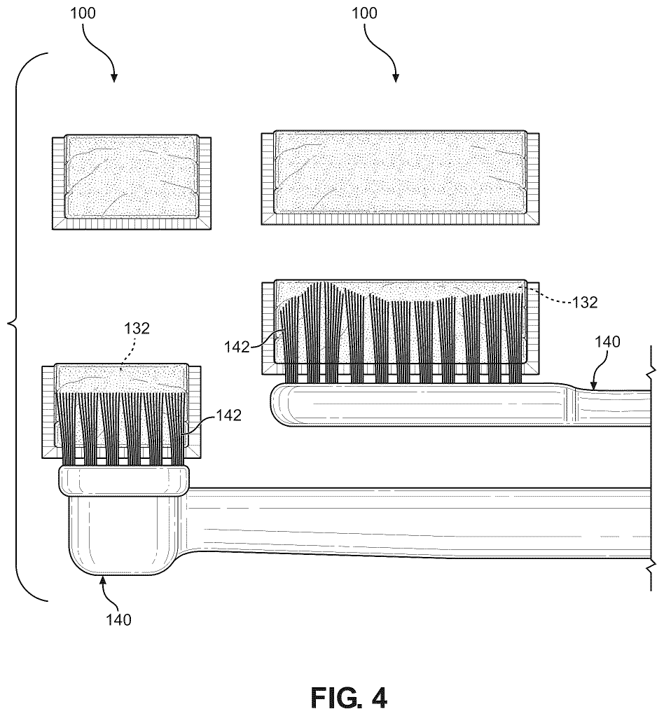

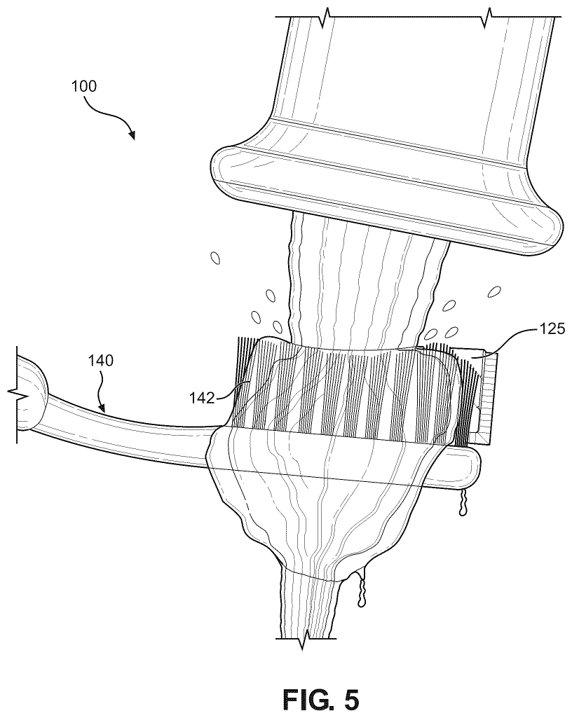

Referring now to FIGS. 1, 4, and 5, there are shown perspective views of alternative embodiments of a toothpaste delivery system 100 in use.

In use, the toothpaste delivery system 100 may be sandwiched in between a plurality of bristles 142 of a toothbrush 140. When the user places the bristles 142 of the toothbrush 140 under water with the toothpaste delivery system 100 therein, the toothpaste delivery system 100 will begin to dissolve. When the user places the bristles 142 of the toothbrush 140 with the toothpaste delivery system 100 into their mouth, the toothpaste delivery system 100 will continue to dissolve. When the toothpaste delivery system 100 may dissolve in the user's mouth, the toothpaste delivery system 100 will release the powdered dentifrice 132. As the user brushes their teeth with the toothbrush 140 and the front layer 110 and the rear layer 120 of the toothpaste delivery system 100 is dissolved, the powdered dentifrice 132 spreads onto the bristles 142 of the toothbrush 140 and thereby onto the user's teeth. When the user completes the brushing process, they will be able to rinse their mouth and the toothbrush 140 to clean away the remnants of the toothpaste delivery system 100 and its contents.

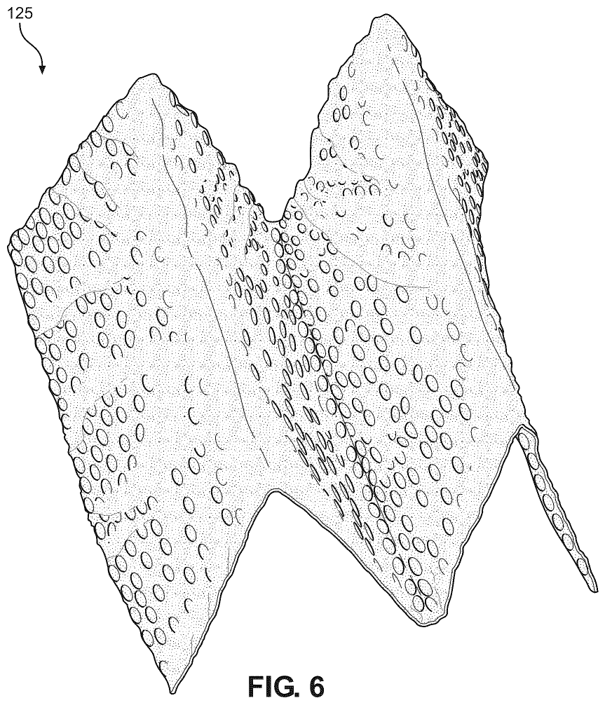

FIG. 6 shows an overhead view of a dental pouch 125 of the toothpaste delivery system 100.

The dental pouch 125 may be textured, dimpled, or have raised diamond shapes for increased surface contact with inside and outside to reduce the volume required inside the dental pouch. The dental pouch 125 may be accordion shaped with the dental pouch 125 having the same ingredients as the dentifrice. The dental pouch 125 may be a single layer or is folded into multiple layers, to increase the amount of dentifrice material to be used.

The foregoing descriptions of specific embodiments of the present invention have been presented for purposes of illustration and description. They are not intended to be exhaustive or to limit the present invention to the precise forms disclosed, and obviously many modifications and variations are possible considering the above teaching. The exemplary embodiment was chosen and described to best explain the principles of the present invention and its practical application, to thereby enable others skilled in the art to best utilize the present invention and various embodiments with various modifications as are suited to the particular use contemplated.

What is claimed as new is:

1. A toothpaste delivery system, comprising:
a front layer;
a rear layer;
wherein the front layer and the rear layer form a dental pouch;
a plurality of seams joining the front layer and the rear layer;
an interior volume formed between the front layer and the rear layer; and
wherein the toothpaste delivery system is adapted to be sandwiched in between a plurality of bristles of a toothbrush.

2. The toothpaste delivery system, according to claim 1, wherein the front layer is composed of a plurality of consumable and dissolvable materials.

3. The toothpaste delivery system, according to claim 1, wherein the front layer is composed of Pullulan.

4. The toothpaste delivery system, according to claim 1, wherein the rear layer is composed of a plurality of consumable and dissolvable materials.

5. The toothpaste delivery system, according to claim 1, wherein the rear layer is composed of Pullulan.

6. The toothpaste delivery system, according to claim 1, wherein the front layer and the rear layer is made from a film used to create the dental pouch.

7. The toothpaste delivery system, according to claim 1, wherein the dental pouch is made of calcium-based material or carbon-based material.

8. The toothpaste delivery system, according to claim 1, wherein the dental pouch is textured, dimpled, or has raised diamond shapes for increased surface contact to the interior volume required inside the dental pouch.

9. The toothpaste delivery system, according to claim 1, wherein the interior volume includes a powdered dentifrice.

10. The toothpaste delivery system, according to claim 9, wherein the powdered dentifrice includes a plurality of carbon activated particles that enhance the powdered dentifrice.

11. The toothpaste delivery system, according to claim 9, wherein the powdered dentifrice limits using excessive toothpaste products.

12. The toothpaste delivery system, according to claim 9, wherein when a user is adapted to brush their teeth with the toothbrush and the front layer and the rear layer of the toothpaste delivery system is dissolved, the powdered dentifrice spreads onto the bristles of the toothbrush and onto a user's teeth.

13. The toothpaste delivery system, according to claim 9, wherein the dental pouch includes a single layer or is folded into multiple layers, to increase the powdered dentifrice used.

14. The toothpaste delivery system, according to claim 9, wherein the dental pouch has the same material as the powdered dentifrice.

15. The toothpaste delivery system, according to claim 1, further comprising a plurality of additives for freshening breath and teeth whitening.

16. The toothpaste delivery system, according to claim 1, wherein the bristles of the toothbrush are placed under water with the toothpaste delivery system to dissolve the toothpaste delivery system onto the bristles of the toothbrush.

17. The toothpaste delivery system, according to claim 1, wherein the bristles of the toothbrush with the toothpaste delivery system are adapted to be placed into a user's mouth, the toothpaste delivery system will continue to dissolve.

18. The toothpaste delivery system, according to claim 11, wherein the bristles of the toothbrush with the toothpaste delivery system are adapted to be placed into the user's mouth, the toothpaste delivery system will release the powdered dentifrice.

19. The toothpaste delivery system, according to claim 13, wherein the user completes brushing their teeth, they will rinse their mouth and the toothbrush to clean away remnants of the toothpaste delivery system.

20. A method for using a toothpaste delivery system, comprising of the steps:
obtaining the toothpaste delivery system;
placing the toothpaste delivery system in between a plurality of bristles of a toothbrush;
brushing a user's teeth with the toothpaste delivery system in between the bristles of the toothbrush;
wetting the toothbrush and dissolving the toothpaste delivery system; and
releasing a powdered dentifrice disposed within the toothpaste delivery system.
